(19) United States
(12) Patent Application Publication
(10) Pub. No.: US 2024/0180596 A1
(43) Pub. Date: Jun. 6, 2024

(54) SYSTEM AND METHOD FOR CORRECTION OF SPINE DEFORMATION

(71) Applicant: Providence Medical Technology, Inc., Pleasanton, CA (US)

(72) Inventors: Bruce M. McCormack, San Francisco, CA (US); Shigeru Tanaka, Half Moon Bay, CA (US)

(21) Appl. No.: 18/284,939

(22) PCT Filed: Mar. 31, 2022
(86) PCT No.: PCT/US2022/022800

§ 371 Date: Sep. 29, 2023

Related U.S. Application Data
(60) Provisional application No. 63/168,342, filed on Mar. 31, 2021.

(51) Int. Cl. A61B 17/70

(52) U.S. Cl. CPC A61B 17/7064; A61B 17/7053; A61B 17/7067

(57) ABSTRACT

An example system includes an anterior cervical discectomy and fusion (ACDF) implant that has a curved inferior surface that allows the vertebral body to rotate at the correct instantaneous center of rotation. An example system may include a one or more surgical instruments to access the cervical facet joint and perform either an osteotomy (partial removal of bone) or a facetectomy (total removal of the facet joint) in order to provide space to move the spinal column and correct kyphotic deformity.

### CROSS REFERENCE TO RELATED APPLICATIONS

This application claims the benefit of priority to U.S. Patent Application 63/168,342, filed Mar. 31, 2021, and titled “System and Method for Correction of Spine Deformation” and which is hereby incorporated by reference in its entirety.

### FIELD

This application relates to systems and methods for correction of spine deformation.

### BACKGROUND

Kyphosis is a convex curvature of the spine that can cause pain and other medical complications for a patient. Osteotomy (partial removal of bone) or a facetectomy (total removal of the facet joint) are two surgical procedures that may be used in order to provide space to move the spinal column and correct kyphotic deformity. Although cervical osteotomy is an established procedure for cervical kyphosis correction, the procedure is not commonly performed outside the university/research hospital setting due to procedural complexity, lack of control and safety concerns. Improved methods and systems to correct spine deformation in patients are needed.

### SUMMARY

Example systems to correct kyphotic deformity are described herein. An example system includes an anterior cervical discectomy and fusion (ACDF) implant that has a curved inferior surface that allows the vertebral body to rotate at the correct instantaneous center of rotation. An example system may include a one or more surgical instruments to access the cervical facet joint and perform either an osteotomy (partial removal of bone) or a facetectomy (total removal of the facet joint) in order to provide space to move the spinal column and correct kyphotic deformity.

In one aspect, a posterior spinous process fixation device to secure the vertebral bodies in the corrected position is described. In one aspect, the most distal spinous process bone is identified. This bone may have the most leverage to reduce a kyphosis but it may be the weakest bone. A spinous process wiring or fixation system disperses the load throughout the spinous process bone, by, for example, including wiring/fixation at the base is stronger than wiring/fixation on the distal spinous process. The wiring/fixation may include a plate feature with teeth to clasp on the spinous process at top and bottom. In another aspect, laminar plates may be used and the plates may have cross links for rotational stability.

An anterior cervical discectomy and fusion (ACDF) implant is disclosed. The implant includes a proximal end portion and a distal end portion. The implant also includes a first surface opposite a second surface, the first surface including one or more teeth or ridges and the second surface being arcuate, curved or dome shaped, each of the first and second surfaces extending between the proximal and distal end portions. The shape of the second surface allows a vertebral body to rotate at a correct center of rotation. In one aspect, the second surface is an inferior surface configured to engage a superior surface of a vertebra. In one aspect, the proximal end portion further comprises an integrated anterior fixation plate extending from the end portion. In one aspect, the anterior fixation plate further comprises at least one opening, and preferably two openings, configured to receive a fixation device. In one aspect, the fixation device is a screw.

A system for correction of spine deformation is disclosed. The system includes the implant as described herein. The system may further include a posterior cervical fixation system to compress a first and second lamina, thereby correcting the spine deformation. In one aspect, the posterior cervical fixation system comprises a laminar hook and rod, or a hook and wire, or a spinous process wiring and fixation device. In one aspect, the system may further include one or more surgical instruments to access the cervical facet joint and perform either an osteotomy (partial removal of bone) or a facetectomy (total removal of the facet joint) in order to provide space to move the spinal column and correct kyphotic deformity.

A method for correction of spine deformation is disclosed. The method includes advancing the implant as disclosed herein into a disc space between an upper and lower vertebrae, the space created in an ACDF procedure. The method further includes fixing the implant to the upper vertebrae thereby allowing the implant to slide or rotate in a controlled manner on a surface of the lower vertebrae following the segment's instantaneous center of rotation during a subsequent posterior reduction osteotomy. In one aspect, the method further includes moving the patient to the prone position. In one aspect, the method further includes removing a lateral mass of the upper and/or lower vertebrae with an access and bone removal device. In one aspect, the method further includes compressing a first lamina toward a second lamina with a compression and fixation system or posterior cervical fixation system to compress the first and second lamina, thereby correcting the kyphotic deformity. The compression and fixation system is a laminar hook/rod, or hook/wire, or a spinous process wiring/fixation device. In some aspects, the first lamina is C5 and the second lamina is C7.

Additional embodiments and features are set forth in part in the description that follows, and will become apparent to those skilled in the art upon examination of the specification of may be learned by the practice of the disclosed subject matter. A further understanding of the nature and advantages of the present disclosure may be realized by reference to the remaining portions of the specification and the drawings, which form a part of this disclosure. One of skill in the art will understand that each of the various aspects and features of the disclosure may advantageously be used separately in some instances, or in combination with other aspects and features of the disclosure in other instances.

### BRIEF DESCRIPTION OF THE DRAWINGS

The description will be more fully understood with reference to the following figures in which components are not drawn to scale, which are presented as various examples of the present disclosure and should not be construed as a complete recitation of the scope of the disclosure, characterized in that:

FIGS. 1A-1B depict isometric and side views of an intervertebral implant according to the disclosure;

FIG. 2A depicts a vertebral column with discs removed at two levels;

FIG. 2B depicts the intervertebral implant inserted at the levels where the disc was removed;

FIG. 2C is an isolated and enlarged view of the surgical site of FIG. 2B;

FIGS. 3A-3B depict removal of bone (osteotomy) at one level;

FIG. 4A depicts the spine before correction, with intervertebral implants according to the disclosure in the surgical site;

FIG. 4B depicts correction of the spine of FIG. 4A;

FIGS. 5A-5C depict another embodiment of a system and method for correction of spinal deformation.

FIG. 6 illustrates a method for correction of a spinal deformity.

### DETAILED DESCRIPTION

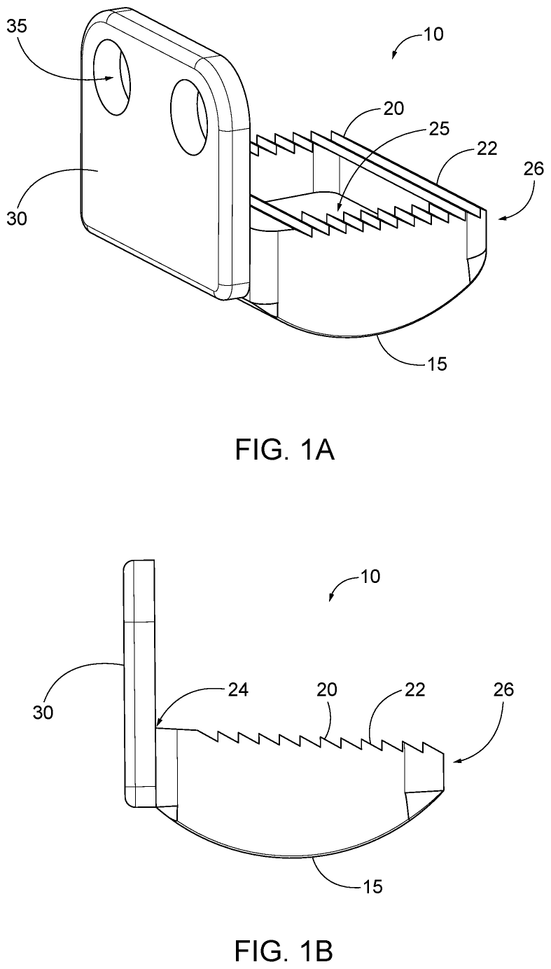

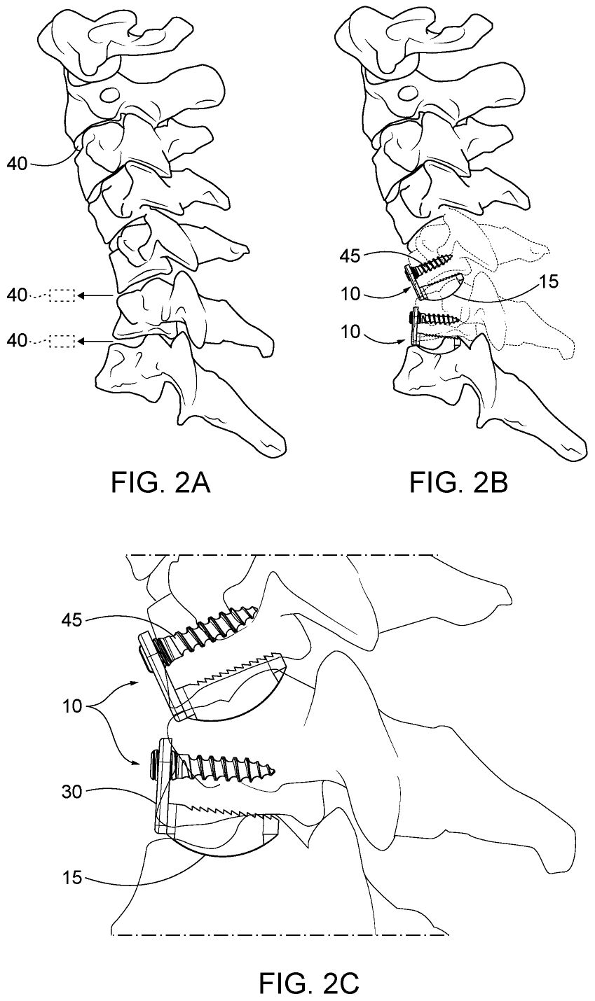

Disclosed herein are methods and systems for correction of spinal deformation. Example systems to correct kyphotic deformity are described herein. As can be understood from FIGS. 1A-1B and others, an example system includes an intervertebral implant 10, such as an anterior cervical discectomy and fusion (ACDF) implant. The implant 10 has a curved, arcuate or domed inferior or second surface 15 to allow rotation on the superior surface of the lower vertebra such that the vertebral body rotates at the correct instantaneous center of rotation when the implant is positioned in the disc space (see e.g. FIGS. 2B-5C). The first or superior surface 20 includes teeth or ridges 22 to engage with a vertebral inferior surface and help to secure the implant and promote fusion. The implant 10 may also include an opening or hollow interior 25 that extends through the first and second surfaces 20, 15 to help promote fusion. In one example, the opening 25 may be a central opening. The implant 10 includes a proximal end portion 24 and a distal end portion 26 and may further include an anterior plate 30. The anterior plate 30 may be integral with a proximal end face or end portion 24 of the implant 10. The anterior plate 30 includes one or more fixation apertures 35 configured to receive a fixation device, such as a screw. An example system may further include a one or more surgical instruments to access the cervical facet joint and perform either an osteotomy (partial removal of bone) or a facetectomy (total removal of the facet joint) in order to provide space to move the spinal column and correct kyphotic deformity.

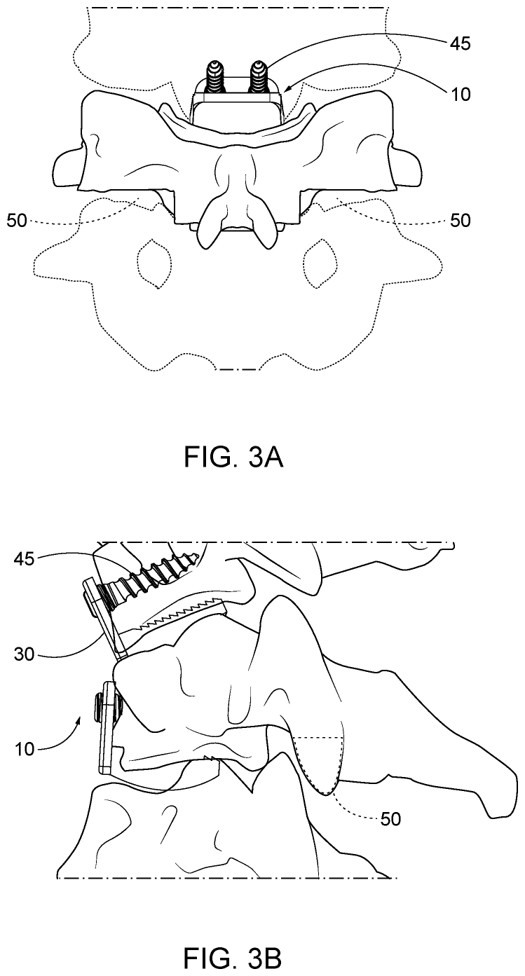

While the steps of the method are disclosed in a specific order, more or fewer steps may be used and the order may change. In one embodiment, and as illustrated in FIGS. 2A-2C, the method begins by performing a standard ACDF exposure. In FIG. 2A, the disc 40 is removed at two levels, typically C5-C7 or C5-T1 depending upon the anatomy of the patient. An implant 10, such as an ACDF intervertebral implant 10, is implanted at the treated levels. In one embodiment, the implant 10 includes an inferior implant surface 15 that is arcuate or curved or dome shaped to allow rotation on the superior surface of the lower vertebra following its instantaneous center of rotation. The implant 10 may include an integrated anterior plate 30 for fixation (such as by a screw 45) into the vertebral body. The anterior plate 30 may be integral with or flush with the proximal end face or portion 24 of the implant 10. As shown in FIGS. 2B and 2C, and others, the implant 10 has a screw fixation feature 45 to the upper vertebra and may not be rigidly fixated to the lower vertebra. This allows the implant 10 to slide and/or rotate in a controlled manner on the lower vertebral surface following the segment's instantaneous center of rotation during the subsequent posterior reduction osteotomy. In a next step of the method, the patient is moved to the prone position. As can be understood from FIGS. 3A-3B, the lateral mass 50 at one level is removed bilaterally (e.g. C6) using a customized access and bone removal instrument. This will qualify as an osteotomy.

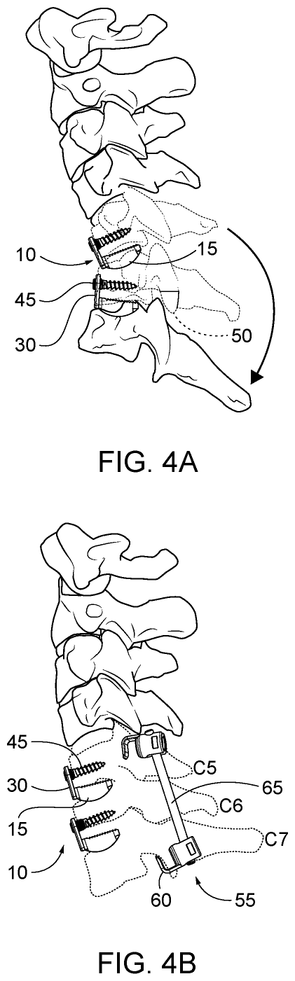

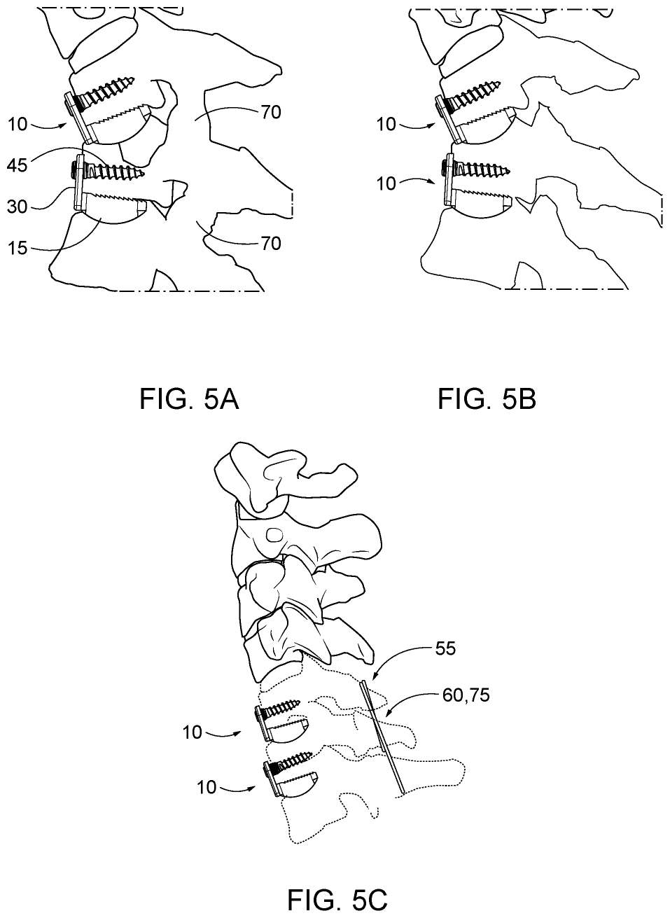

FIG. 4A illustrates the spine of the patient with a kyphotic deformity and the implant 10 at two levels. An osteotomy of the lateral mass has been performed. In other embodiments, a facetectomy or total removal of the facet joint, is performed. See FIGS. 5A-5C. Turning back to FIGS. 4A-4B, in order to correct the spinal deformity, in one example, the C5 lamina is compressed toward the C7 lamina. FIG. 4B depicts the compression and fixation instrumentation or a posterior cervical fixation device 55, such as a laminar hook 60/rod 65 or hook 60/wire or other spinous process wiring/fixation device, which compresses the upper to lower lamina, correcting the kyphotic deformity. In one aspect, the most distal spinous process bone is identified. This bone may have the most leverage to reduce a kyphosis but it may be the weakest bone. A spinous process wiring or fixation system 55 disperses the load throughout the spinous process bone, by, for example, including wiring/fixation at the base is stronger than wiring/fixation on the distal spinous process. The wiring/fixation may include a hook/plate feature with teeth 60 to clasp on the spinous process at top and bottom. In another aspect, laminar plates may be used and the plates may have cross links for rotational stability.

As can be understood from FIGS. 5A-5C, it is also possible to correct kyphosis caused by fusion with the disclosed system. For example, and as shown in FIGS. 5A-5B, at the anterior spine, the method includes removing fused bone 70 if accessible from anterior position and implantation of intervertebral implants 10 at two levels, such as C5-C7. The patient is then turned to a prone position. At the posterior spine, the method includes performing a facetectomy at the treated levels (removal of fused bone 70, shown at FIG. 5A and shown removed at FIG. 5B). As described previously, and shown at FIG. 5C, the kyphotic deformation is corrected by compressing the upper to lower lamina with a posterior cervical fixation device 55, such as a laminar hook 60/rod 65 or hook 60/wire 75 or other spinous process wiring/fixation device and the surgical site (vertebrae) is secured with spinous process fixation instrumentation 55.

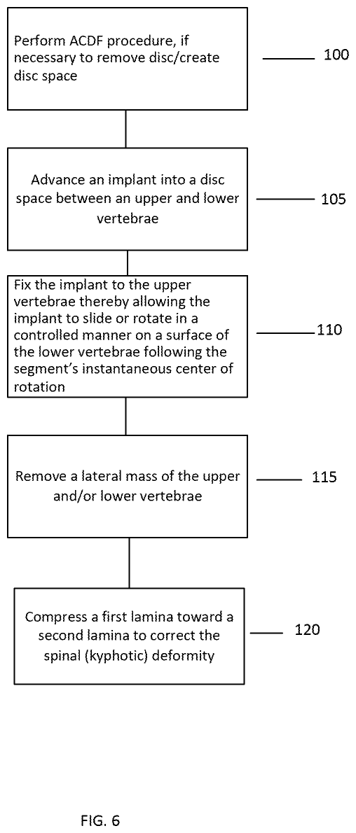

FIG. 6 illustrates a method in accordance with the disclosure. While the steps of the method are disclosed in a specific order, more or fewer steps may be used and the order may change. In step 100, an ACDF procedure is performed, if necessary, to remove a compressed or compromised disc and/or create disc space. In step 105, an implant is advanced into a disc space between an upper and lower vertebrae. In step 110, the implant is fixed to the upper vertebrae thereby allowing the implant to slide or rotate in a controlled manner on a surface of the lower vertebrae following the segment's instantaneous center of rotation. In step 115, a lateral mass of the upper and/or lower vertebrae is removed. In step 120, a first lamina is compressed toward a second lamina to correct the spinal (kyphotic) deformity.

The above specification and examples provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

1. An anterior cervical discectomy and fusion (ACDF) implant comprising:
a proximal end portion and a distal end portion; and
a first surface opposite a second surface, the first surface including one or more teeth or ridges and the second surface being arcuate, curved or dome shaped, each of the first and second surfaces extending between the proximal and distal end portions,
wherein the shape of the second surface allows a vertebral body to rotate at a correct center of rotation.

2. The implant of claim 1, wherein the second surface is an inferior surface configured to engage a superior surface of a vertebra.

3. The implant of claim 1, wherein the proximal end portion further comprises an integrated anterior fixation plate extending from the end portion.

4. The implant of claim 3, wherein the anterior fixation plate further comprises at least one opening, and preferably two openings, configured to receive a fixation device.

5. The implant of claim 4, wherein the fixation device is a screw.

6. A system for correction of spine deformation comprising an ACDF implant, the implant comprising
a proximal end portion and a distal end portion; and
a first surface opposite a second surface, the first surface including one or more teeth or ridges and the second surface being arcuate, curved or dome shaped, each of the first and second surfaces extending between the proximal and distal end portions,
wherein the shape of the second surface allows a vertebral body to rotate at a correct center of rotation.

7. The system of claim 6, further comprising a posterior cervical fixation system to compress a first and second lamina, thereby correcting the spine deformation.

8. The system of claim 7 wherein the posterior cervical fixation system comprises a laminar hook and rod, or a hook and wire, or a spinous process wiring and fixation device.

9. The system of claim 6, further comprising one or more surgical instruments to access the cervical facet joint and perform either an osteotomy (partial removal of bone) or a facetectomy (total removal of the facet joint) in order to provide space to move the spinal column and correct kyphotic deformity.

10. A method for correction of spine deformation comprising:
advancing an ACDF implant of into a disc space between an upper and lower vertebrae, the space created in an ACDF procedure, where the ACDF implant comprises:
a proximal end portion and a distal end portion; and
a first surface opposite a second surface, the first surface including one or more teeth or ridges and the second surface being arcuate, curved or dome shaped, each of the first and second surfaces extending between the proximal and distal end portions,
wherein the shape of the second surface allows a vertebral body to rotate at a correct center of rotation; and
fixing the implant to the upper vertebrae thereby allowing the implant to slide or rotate in a controlled manner on a surface of the lower vertebrae following the segment's instantaneous center of rotation during a subsequent posterior reduction osteotomy.

11. The method of claim 10, further comprising moving the patient to the prone position.

12. The method of claim 10, further comprising removing a lateral mass of the upper and/or lower vertebrae with an access and bone removal device.

13. The method of claim 12 further comprising compressing a first lamina toward a second lamina with a compression and fixation system or posterior cervical fixation system to compress the first and second lamina, thereby correcting the kyphotic deformity.

14. The method of claim 13, wherein the compression and fixation system is a laminar hook/rod, or hook/wire, or a spinous process wiring/fixation device.

15. The method of claim 14, wherein the first lamina is C5 and the second lamina is C7.
